(19) United States
(12) Patent Application Publication
(10) Pub. No.: US 2022/0111254 A1
(43) Pub. Date: Apr. 14, 2022

(54) METHOD FOR BUILDING UP ENERGY METABOLISM SYSTEM TO MONITORING EXERCISE

(71) Applicant: bOMDIC, Inc., New Taipei (TW)

(72) Inventors: PO-CHUN LEE, New Taipei (TW); YU-WEI YU, New Taipei (TW); JR-FANG LIOU, New Taipei (TW); TAI-YU HUANG, New Taipei (TW); CHIEN-YU CHIU, New Taipei (TW)

(21) Appl. No.: 17/070,040

(22) Filed: Oct. 14, 2020

(51) Int. Cl. A63B 24/00; A61B 5/00; A61B 5/11; G16H 20/30

(52) U.S. Cl. CPC A63B 24/0062; A61B 5/4866; G16H 20/30; A61B 5/7267; A61B 5/4519; A61B 5/1118

(57) ABSTRACT

The present invention discloses a method for monitoring an exercise. Acquire a relationship between a degree of active participation of an organism of a human body and an exercise intensity. Build up an energy metabolism system and build up a mathematical model describing that an energy expenditure depends on the exercise intensity and the degree of active participation of the organism of the human body for the energy metabolism system. Determine the degree of active participation of the organism based on the exercise intensity measured in the exercise by the relationship. Use the exercise intensity and the degree of active participation of the organism to estimate the energy expenditure by the mathematical model of the energy metabolism system. Monitor the exercise based on the energy expenditure.

### BACKGROUND OF THE INVENTION

### 1. Field of the Invention

The present invention relates to a method for monitoring an exercise, and more particularly to a method for monitoring an exercise by building up an energy metabolism system.

### 2. Description of Related Art

A combination of many factors must be taken into account in evaluating the exercise condition. The factors may come from the interior of the body or the external environment. Therefore, how to use the limited computer resource (e.g., energy metabolism system) to precisely evaluate exercise condition (e.g., stamina, training load, fatigue or recovery) is very hard.

Conventionally, aerobic energy metabolism system and anaerobic energy metabolism system are both used in the algorithm for evaluating the exercise condition (e.g stamina, training load, fatigue or recovery). The aerobic energy expenditure and the anaerobic energy expenditure are respectively estimated in aerobic energy metabolism system and anaerobic energy metabolism system. Once the energy expenditure resulting from exercise is estimated, how to precisely dividing the energy expenditure into the aerobic energy expenditure and the anaerobic energy expenditure is very important in precisely evaluating exercise condition. Generally, the energy expenditure is divided into the aerobic energy expenditure and the anaerobic energy expenditure mainly based on the algorithm using, the exercise intensity including the parameter of the internal workload (e.g., heart rate) or the parameter of the external workload (e.g., velocity or power). However, the algorithm using the exercise intensity still doesn't reflect real physiological status of the human body.

Accordingly, the present invention proposes a method for monitoring an exercise by building up at energy metabolism system to overcome the above-mentioned disadvantages.

### SUMMARY OF THE INVENTION

The present invention builds up a mathematical model describing that an energy expenditure depends on the exercise intensity and the degree of active participation of the organism of the human body for the energy metabolism system. Compared to the prior art, the algorithm of the mathematical model further uses the degree of active participation of the organism of the human body. The degree of active participation of the organism of the human body is more suitable to be used for evaluating whether the energy metabolism of the organism of the human body is thriving or not than the exercise intensity because the degree of active participation of the organism of the human body is associated with the metabolism of the human body directly (i.e. more directly than the exercise intensity). Therefore, the algorithm of the mathematical model further using the degree of active participation of the organism of the human body can largely reflect real physiological status of the human body so as to further precisely evaluate exercise condition.

In the present invention, the degree of active participation of the organism varying with the exercise intensity is an important technical feature. The physiological status of the human body changes as the exercise intensity changes. For the organism of the human body having the relationship between the degree of active participation of the organism of the human body and the exercise intensity, the degree of active participation also changes as the exercise intensity changes. Therefore, once the physiological status of the human body changes, the algorithm of the mathematical model further using the variable degree of active participation of the organism of the human body can precisely estimate the energy expenditure in step 204 and further precisely evaluate exercise condition in step 205 so as to precisely reflect real-time physiological status of the human body.

In the present invention, that the degree of active participation of each of the components of the organism of the human body may vary with the exercise intensity is an important technical feature. The physiological status of the human body changes as the exercise intensity changes. For each of the components of the organism of the human body having the relationship between the degree of active participation of the component and the exercise intensity, the degree of active participation also changes as the exercise intensity changes. Therefore, once the physiological status of the human body changes, the algorithm of the mathematical model further using the degree of active participation of each of the components of the organism of the human body can precisely estimate the energy expenditure of each energy metabolism system built up for the corresponding component of the organism of the human body based on the degree of active participation AP of each component of the organism of the human body, e.g., in step 404a and in step 404b, (especially, precisely divide the reference energy expenditure into the energy expenditure of each energy metabolism system built up for the corresponding component of the organism of the human body) and further precisely evaluate exercise condition based on the energy expenditure of each energy metabolism system built up for the corresponding component of the organism of the human body, e.g., in step 405 so as to precisely reflect real-time physiological status of the human body.

By the algorithm implemented in the computer of the present invention, the computer of the present invention performs operations described in claims or the following descriptions to building up an energy metabolism system to monitoring an exercise.

In one embodiment, the present invention discloses a method for monitoring an exercise. The method comprises: acquiring a relationship between a degree of active participation of an organism of a human body and an exercise intensity; building up an energy metabolism system and building up a mathematical model describing that an energy expenditure depends on the exercise intensity and the degree of active participation of the organism of the human body for the energy metabolism system; determining the degree of active participation of the organism based on the exercise intensity measured in the exercise by the relationship; using the exercise intensity and the degree of active participation of the organism to estimate the energy expenditure by the mathematical model of the energy metabolism system; and monitoring the exercise based on the energy expenditure.

In one embodiment, the present invention discloses a method for monitoring an exercise. The method comprises: acquiring a first relationship between a first degree of active participation of a first component of an organism of a human body and an exercise intensity; acquiring a second relationship between a second degree of active participation of a second component, of the organism, of the human body and the exercise intensity; building up a first energy metabolism system, and building up a first mathematical model describing that a first energy expenditure depends on the exercise intensity, the first degree of active participation of the first component and the second degree of active participation of the second component for the first energy metabolism system; building up a second energy metabolism system, and building up a second mathematical model describing that a second energy expenditure depends on the exercise intensity, the first degree of active participation of the first component and the second degree of active participation of the second component for the second energy metabolism system; determining the first degree of active participation of the first component based on the exercise intensity measured in the exercise by the first relationship; determining the second degree of active participation of the second component based on the exercise intensity measured in the exercise by the second relationship, using the exercise intensity, the first degree of active participation of the first component and the second degree of active participation of the second component to estimate the first energy expenditure by the first mathematical model of the first energy metabolism system; using the exercise intensity, the first degree of active participation of the first component and the second degree of active participation of the second component to estimate the second energy expenditure by the second mathematical model of the second energy metabolism system; and monitoring the exercise based on the first energy expenditure and the second energy expenditure.

In one embodiment, the present invention discloses a method for monitoring an exercise. The method comprises: acquiring a first relationship between a first degree of active participation of a plurality of hist-switch muscle fibers of a skeletal muscle system of a human body and an exercise intensity measured by a sensor; acquiring a second relationship between a second degree of active participation of a plurality of slow-switch muscle fibers of the skeletal muscle system of the human body and the exercise intensity measured by the sensor; building up a first energy metabolism system, and building up a first mathematical model describing that a first energy expenditure depends on the exercise intensity, the first degree of active participation of the plurality of fast-switch muscle fibers and the second degree of active participation of the plurality of slow-switch muscle fibers for the first energy metabolism system; building up a second energy metabolism system, and building up a second mathematical model describing that a second energy expenditure depends on the exercise intensity, the first degree of active participation of the plurality of fast-switch muscle fibers and the second degree of active participation of the plurality of slow-switch muscle fibers for the second energy metabolism system; determining the first degree of active participation of the plurality of fast-switch muscle fibers based on the exercise intensity measured in the exercise by the first relationship; determining the second degree of active participation of the plurality of slow switch muscle fibers based on the exercise intensity measured in the exercise by the second relationship; using the exercise intensity, the first degree of active participation of the plurality of fast-switch muscle fibers and the second degree of active participation of the plurality of slow-switch muscle fibers to estimate the first energy expenditure by the first mathematical model of the first energy metabolism system; using the exercise intensity, the first degree of active participation of the plurality of fast-switch muscle fibers and the second degree of active participation of the plurality of slow-switch muscle fibers to estimate the second energy expenditure by the second mathematical model of the second energy metabolism system, and monitoring the exercise based on the first energy expenditure and the second energy expenditure.

The detailed technology and above preferred embodiments implemented for the present invention are described in the following paragraphs accompanying the appended drawings for people skilled in the art to well appreciate the features of the claimed invention.

### BRIEF DESCRIPTION OF THE DRAWINGS

The foregoing aspects and many of the accompanying advantages of this invention will become more readily appreciated as the same becomes better understood by reference to the following detailed description when taken in conjunction with the accompanying drawings, wherein:

FIG. 1 illustrates a schematic block diagram of an exemplary apparatus in the present invention;

FIG. 2 illustrates a method for building up an energy metabolism system to monitor an exercise;

FIG. 3 illustrates a schematic block diagram for building up an energy metabolism system to monitor an exercise in FIG. 2;

FIG. 4 further illustrates a method for building up an energy metabolism system to monitor an exercise in one embodiment A₁ of the embodiments A₁ to A_{N} (N is integer and larger than 1) using the method in FIG. 2;

FIG. 5 illustrates a schematic block diagram for building up an energy metabolism system to monitor an exercise in FIG. 4;

FIG. 6A illustrates the relationship between the degree of active participation AP1 of type IIa muscle fibers and the exercise intensity;

FIG. 6B illustrates the relationship between the degree of active participation AP2 of type I muscle fibers and the exercise intensity; and

FIG. 7 illustrates the relationship between the ratio Y and the degree of active participation AP of the organism in one embodiment.

### DETAILED DESCRI ION OF THE ILLUSTRATED EMBODIMENTS

The detailed explanation of the present invention is described as following. The described preferred embodiments are presented for purposes of illustrations and description and they are not intended to limit the scope of the present invention.

Definition of the Terms

The Organism of the Human Body

The organism may be an organic entity. The human body is composed of many biological systems, such as muscular system, respiratory system, digestive system, cardiovascular system, skeletal system and nervous system. The organism may be one of a plurality of biological systems of the human body. The organism nay be also a complete human body. As long as the organism of the human body has a relationship between the degree of active participation of the organism of the human body and the exercise intensity (preferably, the degree of active participation of the organism varies with the exercise intensity), the mathematical model of the enemy metabolism system built up for the organism of the human body can take the relationship into account.

The Degree of Active Participation of the Organism

The degree of active participation of the organism of the human body is a parameter used for evaluating whether the energy metabolism of the organism of the human body is thriving or not. The degree of active participation may be presented in any suitable form. For example, the organism has 100 cells, 80 cells are active, 20 cells are inactive, the degree of active participation is 80% if the degree of active participation is presented in the form of a ratio of the number of the active cells in the organism to the number of the total cells in the organism; the degree of active participation is 4 if the degree of active participation is presented in the form of a ratio of the number of the active cells in the organism to the number of the inactive cells in the organism. For how to distinguish the word “active” from the word “inactive”, an active threshold may be defined such the it is called “active” above the active threshold and it is called “inactive” below the active threshold. The active threshold may be fixed or variable.

Exercise Intensity

The exercise intensity may refer to bow much energy is expended when exercising. The exercise intensity may define how hard the body has to work to overcome a task/exercise. Exercise into may be measured in the form of the internal workload. The parameter of the exercise intensity associated with the internal workload may be associated with a heart rate, an oxygen consumption, a pulse, a respiration rate and RPE (rating perceived exertion). The exercise intensity may be measured in the form of the external workload. The parameter of the exercise intensity associated with the external workload may be associated with a speed, a power, a force, a motion intensity, an energy expenditure rate, a motion cadence or other kinetic data created by the external workload resulting in energy expenditure. The heart rate may be often used as a parameter of the exercise intensity.

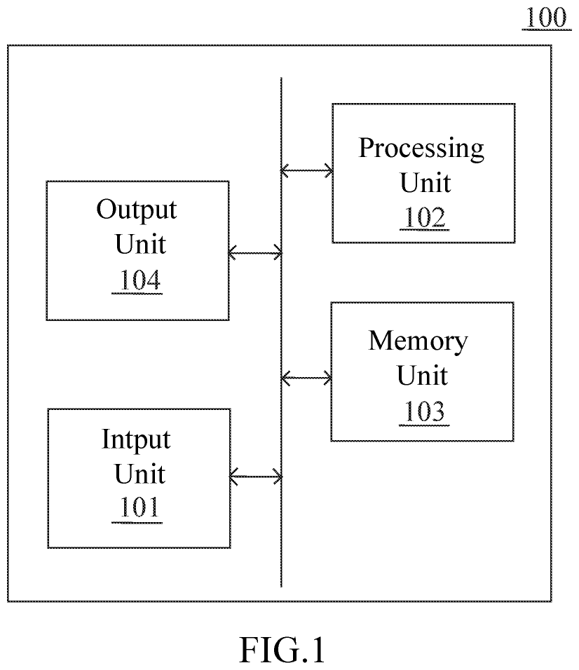

The method in the present invention can be applied in all kinds of apparatuses, such as an exercise measurement system, a wrist top device, a mobile device, a server or a combination of at least one of the exercise measurement system, the wrist top device, the mobile device and the server. FIG. 1 illustrates a schematic block diagram of an exemplary apparatus 100 in the present invention. The apparatus 100 may comprise an input unit 101, a processing unit 102 a memory unit 103 and an output unit 104. The input unit 101 may comprise a first sensor which may measure the exercise intensity associated with the physiological data, the cardiovascular data or the internal workload from the user's body. The exercise intensity may be measured by, applying a skin contact from chest, wrist or any other human part. Preferably, the exercise intensity is a heart rate and the sensor is a heart rate senor. The input unit 101 may comprise a second sensor (e.g., motion sensor) which may measure the exercise intensity associated with the external workload. The second sensor may comprise at least one of an accelerometer, a magnetometer and a gyroscope. The input unit 101 may further comprise a position sensor (e.g., GPS: Global Positioning System). The processing unit 102 may be any suitable processing device for executing software instructions, such as a central processing unit (CPU). The memory unit 103 may include random access memory (RAM) and read only memory (ROM), but it, is not limited to this case. The memory unit 103 may include any suitable non-transitory computer readable medium, such as ROM, CD-ROM, DVD-ROM and so on. Also, the non-transitory computer readable medium is a tangible medium. The non-transitory computer readable medium includes a computer program code which, when executed by the processing unit 102, causes the apparatus 100 to perform desired operations (e.g., operations listed in claims). The output unit 104 may be a display for displaying exercise guiding, exercise scheme or exercise index. The displaying mode may be in the form of words, a voice or an image.

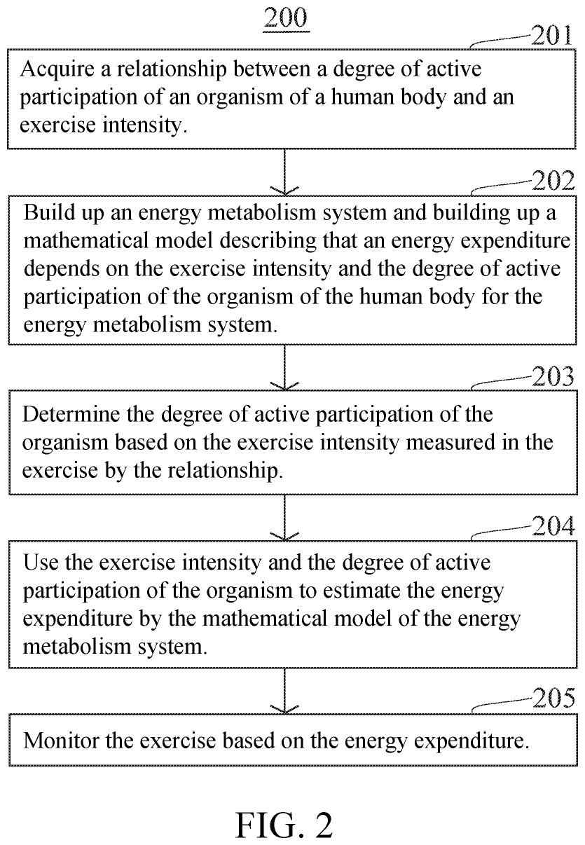

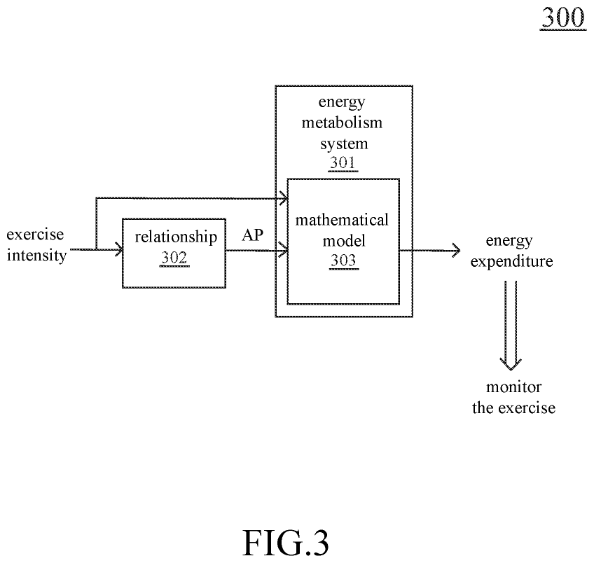

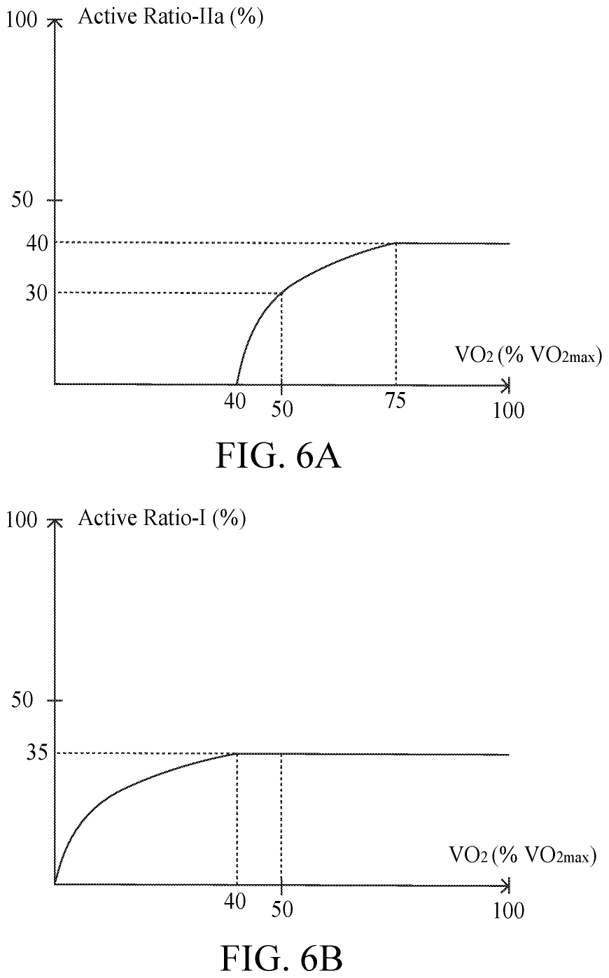

FIG. 2 illustrates a method 200 for building up an energy metabolism system 301 to monitor an exercise. FIG. 3 illustrates a schematic block diagram 300 for building up an energy metabolism system 301 to monitor an exercise in FIG. 2. The process in FIG. 2 starts in step 201: acquiring a relationship 302 between a degree of active participation AP of an organism of a human body and an exercise intensity (from a memory unit 103). The organism may be one of a plurality of biological systems of the human body. The biological system may be a muscular system. The biological system may be a skeletal muscle system. The organism may be also a complete human body. The relationship 302 can be seen in each of FIG. 6A and FIG. 6B. The relationship 302 may be acquired by performing a process. The property of the cell changes when the cell changes from being inactive to being active. For example, when the muscle cells are electrically or neurologically active, the electric potential generated by the muscle cells can be detected by electromyograph. For example, when the cell changes from being inactive to being active, the glycogen reserve in the cell decreases. Therefore, the process capable of detecting the change can be used to acquire the relationship 302.

In Step 202: building up an energy metabolism system 301 and building up a mathematical model 303 describing that an energy expenditure depends on the exercise intensity and the degree of active participation AP of the organism of the human body for the energy metabolism system 301 (by a process unit 102).

In Step 203: determining the degree of active participation AP of the organism based on the exercise intensity measured in the exercise by the relationship 302. Once the exercise intensity is determined, the degree of active participation AP of the organism can be determined by the relationship 302.

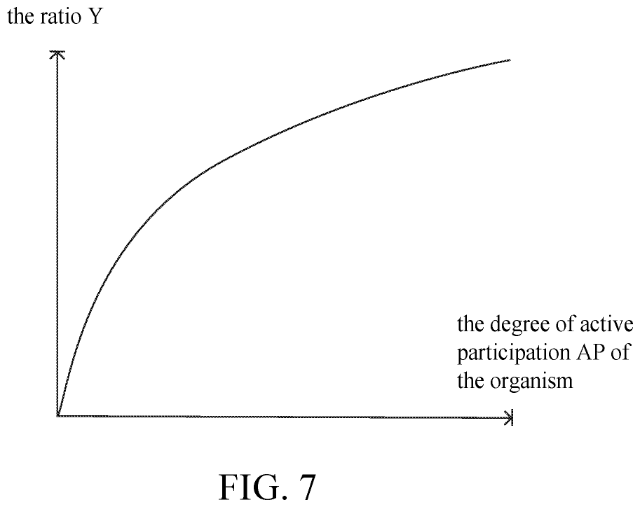

In Step 204: using the exercise intensity and the degree of active participation AP of the organism to estimate the energy expenditure by the mathematical model 303 of the energy metabolism system 301 (by the process unit 102). In one embodiment, determine a reference energy expenditure based on the exercise intensity; and estimate the energy expenditure based on the degree of active participation AP of the organism and the reference energy expenditure. The reference energy expenditure may be an additional energy expenditure of the human body resulting from the exercise, so the reference energy expenditure may exclude the basic metabolism energy of the human body. The reference energy expenditure may be determined by any suitable method. For example, if the exercise intensity is presented in the form of the velocity, the reference energy expenditure may be acquired by the formula: the reference energy expenditure=0.5*the mass of the human body*velocity². If the exercise intensity is presented in the form of the energy expenditure rate, the reference energy expenditure may be acquired by the formula: the reference energy expenditure=the energy expenditure rate*exercise time; however, the present invention is not limited to these cases. The energy expenditure may be estimated by the formula: the reference energy expenditure*ratio Y; the ratio Y may be adjusted based on the degree of active participation AP of the organism, or the ratio Y may be adjusted based on a combination of the degree of active participation AP of the organism and any other associated parameter. The relationship between the ratio Y and the degree of active participation AP of the organism may be shown in FIG. 7 according to the observation or the result derived from the algorithm.

In Step 205: monitoring the exercise based on the energy expenditure (by the process unit 102). The energy metabolism system 301 may have an energy reserve, wherein the exercise is monitored based on a ratio of the energy expenditure to the energy reserve. The details may be shown in step 405. Monitoring the exercise may comprise estimating the exercise-monitoring parameters based on the energy expenditure and displaying words, a voice or an image generated based on the exercise-monitoring parameters to remind the user taking exercise by the output unit 104 of the electronic apparatus 100. Monitoring the exercise may comprise estimating the exercise-monitoring parameters based on the energy expenditure and providing exercise guiding or exercise suggestion for the user taking exercise. The exercise-monitoring parameters may comprise stamina, training load, injury risk, fatigue or recovery.

In the present invention, the degree of active participation AP of the organism varying with the exercise intensity is an important technical feature. The physiological status of the human body changes as the exercise intensity changes. For the organism of the human body having the relationship between the degree of active participation AP of the organism of the human body and the exercise intensity, the degree of active participation also changes as the exercise intensity changes. Therefore, once the physiological status of the human body changes, the algorithm of the mathematical model further using the variable degree of active participation AP of the organism of the human body can precisely estimate the energy expenditure in step 204 and further precisely evaluate exercise condition in step 205 so as to precisely reflect real-time physiological status of the human body.

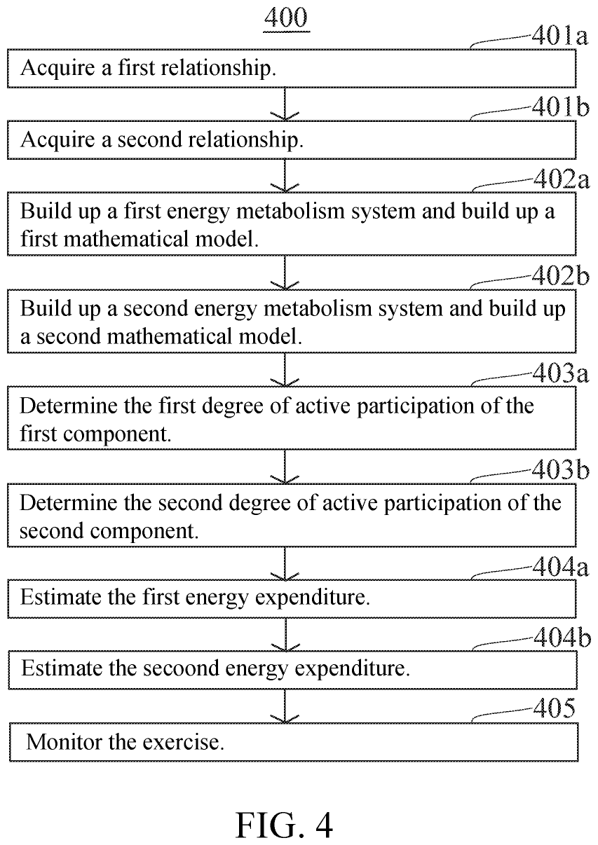

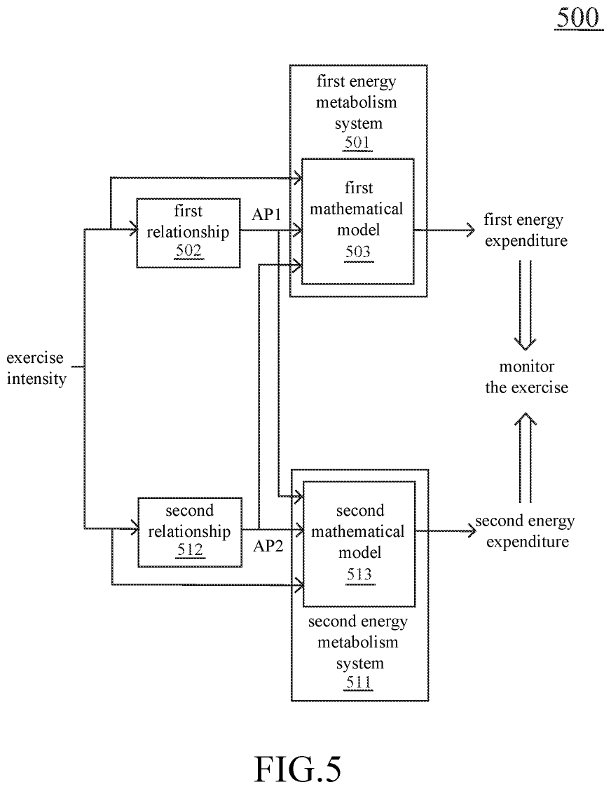

FIG. 4 further illustrates a method 400 for building up an energy metabolism system 501 to monitor an exercise in one embodiment A₁ of the embodiments A₁ to A_{N} (N is integer and larger than 1) using the method 200 in FIG. 2. FIG. 5 illustrates a schematic block diagram 500 for building up an energy metabolism system 501 to monitor an exercise in FIG. 4.

The process in FIG. 4 starts in step 401a and step 401b. In step 401a, acquire a first relationship 502 between a first degree of active participation AP1 of a first component of an organism of a human body and an exercise intensity (from a memory unit 103). In step 401b: acquiring a second relationship 512 between a second degree of active participation AP2 of a second component of the organism of the human body and the exercise intensity (from the memory unit 103). The organism may be one of a plurality of biological systems of the human body. The biological system may be a muscular system. The biological system may be a skeletal muscle system. The first relationship 502 and the second relationship 512 can be respectively seen in FIG. 6A and FIG. 6B. Each of the first relationship 502 and the second relationship 512 may be acquired by performing a process. The property of the cell changes when the cell changes from being inactive to being active. For example, when the muscle cells are electrically or neurologically active, the electric potential generated by the muscle cells can be detected by electromyograph. For example, when the cell changes from being inactive to being active, the glycogen reserve in the cell decreases. Therefore, a process capable of detecting the change can be used to acquire each of the first relationship 502 and the second relationship 512.

In step 402a: build up a first energy metabolism system 501, and build up a first mathematical model 503 describing that a first energy expenditure depends on the exercise intensity, the first degree of active participation AP1 of the first component and the second degree of active participation AP2 of the second component for the first energy metabolism system 501 (by a process unit 102). In step 402b: build up a second energy metabolism system 511, and build up a second mathematical model 513 describing that a second energy expenditure depends on the exercise intensity, the first degree of active participation AP1 of the first component and the second degree of active participation AP2 of the second component for the second energy metabolism system 511 (by the process unit 102). The first threshold of the exercise intensity, above which the first energy metabolism system 501 is operated, may be larger than the second threshold of the exercise intensity, above which the second energy metabolism system 511 is operated.

In step 403a: determine the first degree of active participation AP1 of the first component based on the exercise intensity measured in the exercise by the first relationship 502. In step 403b: determine the second degree of active participation AP2 of the second component based on the exercise intensity measured in the exercise by the second relationship 512. Once the exercise intensity is determined, the first degree of active participation AP1 of the first component and the second degree of active participation AP2 can be respectively determined by the first relationship 502 and the second relationship 512.

In step 404a: use the exercise intensity, the first degree of active participation AP1 of the first component and the second degree of active participation AP2 of the second component, to estimate the first energy expenditure by the first mathematical model 503 of the first energy metabolism system 501 (by the process unit 102). In step 404b: use the exercise intensity, the first degree of active participation AP1 of the first component and the second degree of active participation AP2 of the second component to estimate the second energy expenditure by the second mathematical model 513 of the second energy metabolism system 511 (by the process unit 102). In one embodiment, determine a reference energy expenditure based on the exercise intensity; and estimate the first energy expenditure and the second energy expenditure based on the first degree of active participation AP1 of the first component, the second degree of active participation AP2 of the second component and the reference energy expenditure. Each of the first energy expenditure and the second energy expenditure may be estimated based on a ratio of the first degree of active participation AP1 of the first component to the second degree of active participation AP2 of the second component. Each of the first energy expenditure and the second energy expenditure may be estimated based on a ratio of the first degree of active participation AP1 of the first component to the second degree of active participation AP2 of the second component and the reference energy expenditure. The reference energy expenditure may be an additional energy expenditure of the human body resulting from the exercise, so the reference energy expenditure may exclude the basic metabolism energy of the human body. The reference energy expenditure may be determined by any suitable method. For example, if the exercise intensity is presented in the form of the velocity, the reference energy expenditure may be acquired by the formula: the reference energy expenditure=0.5*the mass of the human body*velocity². If the exercise intensity is presented in the form of the energy expenditure rate, the reference energy expenditure may be acquired by the formula: the reference energy expenditure=the energy expenditure rate*exercise time; however, the present invention is not limited to these cases.

In step 405; monitor the exercise based on the first energy expenditure and the second energy expenditure (by the process unit 102). The first energy metabolism system 501 may have a first energy reserve and the second energy metabolism system 511 may have a second energy reserve, wherein the exercise is monitored based on a first ratio of the first energy expenditure to the first energy reserve and a second ratio of the second energy expenditure to the second energy reserve. Monitoring the exercise may comprise estimating the exercise-monitoring parameters based on the energy expenditure (the first energy expenditure and the second energy expenditure) and displaying words, a voice or an image generated based on the exercise-monitoring parameters to remind the user taking exercise by the output unit 104 of the electronic. apparatus 100. Monitoring the exercise may comprise estimating the exercise-monitoring parameters based on the energy expenditure (the first energy expenditure and the second energy expenditure) and providing exercise guiding or exercise suggestion for the user taking exercise. The exercise-monitoring parameters may comprise stamina, training load, injury risk, fatigue or recovery.

The following specifically describes each step in FIG. 4.

The method 400 in FIG. 4 is one embodiment A₁ of the embodiments A₁ to A_{N} (N is integer and larger than 1) using the method 200 in FIG. 2. The first energy metabolism system 501 in FIG. 5 corresponds to the energy metabolism system 301 in FIG. 3; the first relationship 502 in FIG. 5 corresponds to the relationship 302 in FIG. 3; the first mathematical model 503 in FIG. 5 corresponds to the mathematical model 303 in FIG. 3. Step 401a in FIG. 4 corresponds to step 201 in FIG. 2; step 402a in FIG. 4 corresponds to step 202 in FIG. 2; step 403a of FIG. 4 corresponds to step 203 in FIG. 2; step 404a in FIG. 4 corresponds to step 204 in FIG. 2; step 405 in FIG. 4 corresponds to step 205 in FIG. 2.

For convenience to describe the embodiment A₁ using the method 400 in FIG. 4, the embodiment A₁ using the method 400 in FIG. 4 further comprises an second energy metabolism system 511; the first mathematical model 503 of the first energy metabolism system 501 and the second mathematical model 513 of the second energy metabolism system 511 are respectively built up based on a first component and a second component of an organism of a human body; besides, the organism in the embodiment A₁ is a skeletal muscle system which is one sub-system of the muscular system, and the first component and the second component are respective type IIa muscle fibers and type I muscle fibers (The skeletal muscle system has two types: slow-switch muscle fibers and fast-switch muscle fibers; slow-switch muscle fibers have one type: type muscle fibers, and fast-switch muscle fibers have two types: type Ila muscle fibers and type IIx muscle fibers). It should be noted that the above arrangement in the embodiment A₁ is merely for convenience of description; however, the present invention is not limited to this case.

In the embodiment A₁, the feature that the degree of active participation AP of the organism varies with the exercise intensity in the skeletal muscle system of the human body is used as the energy metabolism feature associated with the mathematical model of the energy metabolism system; however, as long as any other organism of the human body has this feature, the feature can be used as the energy metabolism feature associated with the mathematical model of the energy metabolism system for it.

Acquire the relationship 502 between the degree of active participation AP1 of type IIa muscle fibers and the exercise intensity (step 401a). Acquire the relationship 512 between the second degree of active participation AP2 of type I muscle fibers and the exercise intensity (step 401b). Type I muscle fibers, type IIa muscle fibers and type IIx muscle fibers are called the overall muscle fibers. FIG. 6A illustrates the relationship 502 between the degree, of active participation AP1 of type IIa muscle fibers and the exercise intensity. In FIG. 6A, the parameter of the degree of active participation AP1 of type IIa muscle fibers is a ratio of the number of active type IIa muscle fibers to the number of the overall muscle fibers abbreviated as Active Ratio-IIa and the parameter of the exercise intensity is the oxygen consumption abbreviated as VO₂. The unit of Active Ratio-IIa is and the unit of VO₂ is VO₂ₘₐₓ. Type IIa muscle fibers are active above about 40% VO₂ₘₐₓ and the maximum of Active Ratio-IIa is about 40%. FIG. 6B illustrates the relationship 512 between the degree of active participation AP2 of type I muscle fibers and the exercise intensity. In FIG. 6B, the parameter of the degree of active participation AP2 of type I muscle fibers is a ratio of the number of active type I muscle fibers to the number of the overall muscle fibers abbreviated as Active Ratio-I and the parameter of the exercise intensity is the oxygen consumption abbreviated as VO₂. The unit of Active Ratio-I is % and the unit of VO₂ is % VO₂ₘₐₓ. Type I muscle fibers are active above about 0% VO₂ₘₐₓ and the maximum of Active Ratio-I is about 35%. Each of the relationship 502 in FIG. 6A and the relationship 512 in FIG. 6B may be acquired by performing a process which has been described previously.

Build up the energy metabolism system 501 of type IIa muscle fibers, and build up the mathematical model 503 describing that the first energy expenditure depends on the exercise intensity, the degree of active participation AP1 of type IIa muscle fibers and the degree of active participation AP2 of type I muscle fibers for energy metabolism system 501 of type IIa muscle fibers (step 402a). Build up the energy metabolism system 511 of type I muscle fibers, and build up the mathematical model 513 describing that the second energy expenditure depends on the exercise intensity, the degree of active participation AP1 of type Ila muscle fibers and the degree of active participation AP2 of type I muscle fibers for the energy metabolism system 511 of type I muscle fibers (step 402b).

Determine the degree of active participation AP1 of type Ila muscle fibers based on the exercise intensity measured in the exercise by the relationship 502 in FIG. 6A (step 403a). Determine the degree of active participation AP2 of type I muscle fibers based on the exercise intensity measured in the exercise by the relationship 512 in FIG. 6B (step 403b). Once the exercise intensity is determined, the degree of active participation AP1 of type IIa muscle fibers and the degree of active participation AP2 of type I muscle fibers are respectively determined by the relationship 502 in FIG. 6A and the relationship 512 in FIG. 6B.

Use the exercise intensity, the degree of active participation AP1 of type IIa muscle fibers and the degree of active participation AP2 of type I muscle fibers to estimate the first energy expenditure by the mathematical model 503 of the energy metabolism system 501 of type Ila muscle fibers (step 404a). Use the exercise intensity, the degree of active participation AP1 of type IIa muscle fibers and the degree of active participation AP2 of type I muscle fibers to estimate the second energy expenditure by the mathematical model 513 of the energy metabolism system 511 of type I muscle fibers (step 404b). In one embodiment, determine a reference energy expenditure based on the exercise intensity; and estimating the first energy expenditure and the second energy expenditure based on the degree of active participation AP1 of type IIa muscle fibers, the degree of active participation AP2 of type I muscle fibers and the reference energy expenditure. For example, each of the first energy expenditure and the second energy expenditure may be estimated based on a ratio of the degree of active participation AP1 of type IIa muscle fibers to the degree of active participation AP2 of type I muscle fibers. Take a simple example (I), see FIG. 6A and FIG. 6B; when the exercise intensity is 50% VO₂ₘₐₓ, Active Ratio-IIa is about 30%, Active Ratio-I is about 35% (i.e. the maximum of Active Ratio-I) and the ratio of Active Ratio-IIa to Active Ratio-I is 30/35; when the user having a mass of 60 kg runs at the velocity (i.e. the exercise intensity) of 8 m/s, the reference energy expenditure is 1920 J (0.5*60*8²); if the reference energy expenditure is divided into the first energy expenditure and the second energy expenditure based on 30/35 (i.e. the ratio of Active Ratio-IIa to Active Ratio-I), the first energy expenditure is about 886 J (1920*30/(30+35)) and the second energy expenditure is about 1034 J (1920*35/(30+35)); it should be noted that the present invention is not limited to this case and comprise any other more complicated case.

Monitor the exercise based on the first energy expenditure and the second energy expenditure (step 405). Take estimating stamina (determined by taking U.S. application Ser. No. 14/718,104 as a reference) for example: the energy metabolism system of type Ila muscle fibers has an energy reserve 5000 J and the energy metabolism system of type I muscle fibers has an energy reserve 10000 J; if the first energy expenditure is about 886 J and the second energy expenditure is about 1034 J by taking example (I) as a reference, the first remaining energy ratio of the energy metabolism system of type IIa muscle fibers is 82.28% (1−886/5000) and the second remaining energy ratio of the energy metabolism system of type muscle fibers is 89.66% (1−1034/10000); the stamina may be a function of the first remaining energy ratio R₁ and the second remaining energy ratio R₂, such as c1*R₁+c2*R₂ (each of the coefficients c1, c2 is positive, and each of the coefficients c1, c2 may be fixed or variable according to the observation of the physiological phenomenon).

Although the first component and the second component in the embodiment A₁ are respectively type IIa muscle fibers and type I muscle fibers of the skeletal muscle system, the first component and the second component in the present invention may be respectively fast-switch muscle fibers and slow-switch muscle fibers of the skeletal muscle system, or type IIx muscle fibers and type IIa muscle fibers of the skeletal muscle system or type IIx muscle fiber and type I muscle fibers of the skeletal muscle system.

In the present invention, that the degree of active participation AP of each of the components of the organism of the human body may vary with the exercise intensity is an important technical feature. The physiological status of the human body changes as the exercise intensity changes. For each of the components of the organism of the human body having the relationship between the degree of active participation AP of the component and the exercise intensity, the degree of active participation AP also changes as the exercise intensity changes. Therefore, once the physiological status of the human body changes, the algorithm of the mathematical model further using the degree of active participation AP of each of the components of the organism of the human body can body can precisely estimate the energy expenditure of each energy metabolism system built up for the corresponding component of the organism of the human body based on the degree of active participation AP of each component of the organism of the human body, e.g., in step 404a and in step 404b, (especially, precisely divide the reference energy expenditure into the energy expenditure of each energy metabolism system built up for the corresponding component of the organism of the human body) and further precisely evaluate exercise condition based on the energy expenditure of each energy metabolism system built up for the corresponding component of the organism of the human body, e.g., in step 405 so as to precisely reflect real-time physiological status of the human body.

The above disclosure is related to the detailed technical contents and inventive features thereof. People skilled in the art may proceed with a variety of modifications and replacements based on the disclosures and suggestions of the invention as described without departing from the characteristics thereof. Nevertheless, although such modifications and replacements are not fully disclosed in the above descriptions, they have substantially been covered in the following claims as appended.

What is claimed is:

1. A method for monitoring an exercise, comprising:
acquiring, from a memory unit, a relationship between a degree of active participation of an organism of a human body and an exercise intensity;
building up, by a process unit, an energy metabolism system and building up, by the process unit, a mathematical model describing that an energy expenditure depends on the exercise intensity and the degree of active participation of the organism of the human body for the energy metabolism system;
determining the degree of active participation of the organism based on the exercise intensity measured in the exercise by the relationship;
using, by the process unit, the exercise intensity and the degree of active participation of the organism to estimate the energy expenditure by the mathematical model of the energy metabolism system; and
monitoring, by the process unit, the exercise based on the energy expenditure.

2. The method according to claim 1, wherein the degree of active participation of the organism varies with the exercise intensity.

3. The method according to claim 1, wherein the energy metabolism system has an energy reserve, wherein the exercise is monitored based on a ratio of the energy expenditure to the energy reserve.

4. The method according to claim 1, wherein the relationship is acquired by performing a process.

5. The method according to claim 4, wherein the process is capable of detecting that a property of a cell changes when the cell changes from being inactive to being active.

6. The method according to claim 1, wherein the exercise intensity is an energy expenditure rate.

7. The method according to claim 1, wherein using the exercise intensity and the degree of active participation of the organism to estimate the energy expenditure by the mathematical model of energy metabolism system comprises:
determining a reference energy expenditure based on the exercise intensity; and
estimating the energy expenditure based on the degree of active participation of the organism and the reference energy expenditure.

8. The method according to claim 1, wherein the organism is a first biological system being one of a plurality of biological systems of the human body.

9. The method according to claim 8, wherein the first biological system is a skeletal muscle system.

10. A method for monitoring an exercise, comprising:
acquiring, from a memory unit, a first relationship between a first degree of active participation of a first component of an organism of a human body and an exercise intensity;
acquiring, from the memory unit, a second relationship between a second degree of active participation of a second component of the organism of the human body and the exercise intensity;
building up, by a process unit, a first energy metabolism system, and building up a first mathematical model describing that a first energy expenditure depends on the exercise intensity, the first degree of active participation of the first component and the second degree of active participation of the second component for the first energy metabolism system;
building up, by the process unit, a second energy metabolism system, and building up a second mathematical model describing that a second energy expenditure depends on the exercise intensity, the first degree of active participation of the first component and the second degree of active participation of the second component for the second energy metabolism system;
determining the first degree of active participation of the first component based on the exercise intensity measured in the exercise by the first relationship;
determining the second degree of active participation of the second component based on the exercise intensity measured in the exercise by the second relationship;
using, by the process unit, the exercise intensity, the first degree of active participation of the first component and the second degree of active participation of the second component to estimate the first energy expenditure by the first mathematical model of the first energy metabolism system;
using, by the process unit, the exercise intensity, the first degree of active participation of the first component and the second degree of active participation of the second component to estimate the second energy expenditure by the second mathematical model of the second energy metabolism system; and
monitoring, by the process unit, the exercise based on the first energy expenditure and the second energy expenditure.

11. The method according to claim 10, wherein each of the first degree of active participation of the first component and the second degree of active participation of the second component varies with the exercise intensity.

12. The method according to claim 10, wherein the first energy metabolism system has a first energy reserve and the second energy metabolism system has a second energy reserve, wherein the exercise is monitored based on a first ratio of the first energy expenditure to the first energy reserve and a second ratio of the second energy expenditure to the second energy reserve.

13. The method according to claim 10, wherein each of the first relationship and the second relationship is acquired by performing a process.

14. The method according to claim 13, wherein the process is capable of detecting that a property of a cell changes when the cell changes from being inactive to being active.

15. The method according to claim 10, wherein the exercise intensity is an energy expenditure rate.

16. The method according to claim 10, wherein using the exercise intensity, the first degree of active participation of the first component and the second degree of active participation of the second component to estimate the first energy expenditure by the first mathematical model of the first energy metabolism system and to estimate the second energy expenditure by the second mathematical model of the second energy metabolism system comprises:
determining a reference energy expenditure based on the exercise intensity; and
estimating the first energy expenditure and the second energy expenditure based on the first degree of active participation of the first component, the second degree of active participation of the second component and the reference energy expenditure.

17. The method according to claim 10, wherein each of the first energy expenditure and the second energy expenditure is estimated based on a ratio of the first degree of active participation of the first component to the second degree of active participation of the second component.

18. The method according to claim 10, wherein the first, energy metabolism system is operated above a first threshold of the exercise intensity and the second energy metabolism system is operated above a second threshold of the exercise intensity, wherein the first threshold of the exercise intensity is larger than the second threshold of the exercise intensity.

19. The method according to claim 10, wherein the exercise intensity is measured by a sensor.

20. A method for monitoring an exercise, comprising:
acquiring, from a memory unit, a first relationship between a first degree of active participation of a plurality of fast-switch muscle fibers of a skeletal muscle system of a human body, and an exercise intensity measured by a sensor;
acquiring, from the memory unit, a second relationship between a second degree of active participation of a plurality of slow-switch muscle fibers of the skeletal muscle system of the human body and the exercise intensity measured by the sensor;
building up, by a process unit, a first energy metabolism system, and building up a first mathematical model describing that a first energy expenditure depends on the exercise intensity, the first degree of active participation of the plurality of fast-switch muscle fibers and the second degree of active participation of the plurality of slow-switch muscle fibers for the first energy metabolism system;
building up, by the process unit, a second energy metabolism system, and building up a second mathematical model describing that a second energy expenditure depends on the exercise intensity, the first degree of active participation of the plurality of fast-switch muscle fibers and the second degree of active participation of the plurality of slow-switch muscle fibers for the second energy metabolism system;
determining the first degree of active participation of the plurality of fast-switch muscle fibers based on the exercise intensity measured in the exercise by the first relationship;
determining the second degree of active participation of the plurality of slow-switch muscle fibers based on the exercise intensity measured in the exercise by the second relationship;
using, by the process unit, the exercise intensity, the first degree of active participation of the plurality of fast-switch muscle fibers and the second degree of active participation of the plurality of slow-switch muscle fibers to estimate the first energy expenditure by the first mathematical model of the first energy metabolism system;
using, by the process unit, the exercise intensity, the first degree of active participation of the plurality of fast-switch muscle fibers and the second degree of active participation of the plurality of slow-switch muscle fibers to estimate the second energy expenditure by the second mathematical model of the second energy metabolism system; and
monitoring, by the process unit, the exercise based on the first energy expenditure and the second energy expenditure.
